SEQUENCE LISTING (28 sequences)

SEQ ID NO: 1 - skipped

SEQ ID NO: 2 - protein, 5 aa, synthetic construct
  SITE 4: note = X may be amino acid
VPGXG

SEQ ID NO: 3 - protein, 167 aa, synthetic construct
MKGSGVGGVGVGGVGVGGVGVGGVGVGGVGKLVPGVGVPGVGVPGFGVPGVGVPGVGVPGVGVPGVGVPGFGVPGVGVPGVGVPGVGVPGVGVPGFGVPGVGVPGVGVPGVGVPGVGVPGFGVPGVGVPGVGVPGVGVPGVGVPGFGVPGVGVPGVGLWLEHHHHHH

SEQ ID NO: 4 - protein, 56 aa, synthetic construct
MKGSGVGGVGVGGVGVGGVGVGGVGVGGVGVPGVGVPGVGVPGFGVPGVGVPGVGC

SEQ ID NO: 5 - protein, 81 aa, synthetic construct
MKGSGVGGVGVGGVGVGGVGVGGVGVGGVGVPGVGVPGVGVPGFGVPGVGVPGVGVPGVGVPGVGVPGFGVPGVGVPGVGC

SEQ ID NO: 6 - protein, 35 aa, synthetic construct
VPGVGVPGVGVPGFGVPGVGVPGVGLWLEHHHHHH

SEQ ID NO: 7 - protein, 60 aa, synthetic construct
VPGVGVPGVGVPGFGVPGVGVPGVGVPGVGVPGVGVPGFGVPGVGVPGVGLWLEHHHHHH

SEQ ID NO: 8 - protein, 85 aa, synthetic construct
VPGVGVPGVGVPGFGVPGVGVPGVGVPGVGVPGVGVPGFGVPGVGVPGVGVPGVGVPGVGVPGFGVPGVGVPGVGLWLEHHHHHH

SEQ ID NO: 9 - protein, 6 aa, synthetic construct
LWLGSG

SEQ ID NO: 10 - protein, 4 aa, synthetic construct
RGDS

SEQ ID NO: 11 - protein, 8 aa, synthetic construct
LPGFPGLK

SEQ ID NO: 12 - protein, 8 aa, synthetic construct
LPGFPGTP

SEQ ID NO: 13 - protein, 9 aa, synthetic construct
GPPGLSGPP

SEQ ID NO: 14 - protein, 8 aa, synthetic construct
FPGPPGPP

SEQ ID NO: 15 - protein, 8 aa, synthetic construct
LPGLPGPP

SEQ ID NO: 16 - protein, 8 aa, synthetic construct
FPGLPGPP

SEQ ID NO: 17 - protein, 10 aa, synthetic construct
GPPGPPGSPG

SEQ ID NO: 18 - protein, 8 aa, synthetic construct
LPGPPGPP

SEQ ID NO: 19 - protein, 9 aa, synthetic construct
FPGSPGFPG

SEQ ID NO: 20 - protein, 9 aa, synthetic construct
GSPGLPGTP

SEQ ID NO: 21 - protein, 8 aa, synthetic construct
IGLSGEKG

SEQ ID NO: 22 - protein, 4 aa, synthetic construct
REDV

SEQ ID NO: 23 - protein, 56 aa, synthetic construct
MKGSGVPGVGVPGVGVPGFGVPGVGVPGVGVPGVGVPGVGVPGFGVPGVGVPGVGC

SEQ ID NO: 24 - protein, 81 aa, synthetic construct
MKGSGVPGVGVPGVGVPGFGVPGVGVPGVGVPGVGVPGVGVPGFGVPGVGVPGVGVPGVGVPGVGVPGFGVPGVGVPGVGC

SEQ ID NO: 25 - protein, 76 aa, synthetic construct
MKGSGVGGVGVGGVGVGGVGVGGVGVPGVGVPGVGVPGFGVPGVGVPGVGVPGVGVPGVGVPGFGVPGVGVPGVGC

SEQ ID NO: 26 - protein, 60 aa, synthetic construct
VPGVGVPGVGVPGFGVPGVGVPGVGVGGVGVGGVGVGGVGVGGVGVGGVGLWLEHHHHHH

SEQ ID NO: 27 - protein, 80 aa, synthetic construct
VPGVGVPGVGVPGFGVPGVGVPGVGVPGVGVPGVGVPGFGVPGVGVPGVGVGGVGVGGVGVGGVGVGGVGLWLEHHHHHH

SEQ ID NO: 28 - protein, 85 aa, synthetic construct
VPGVGVPGVGVPGFGVPGVGVPGVGVPGVGVPGVGVPGFGVPGVGVPGVGVGGVGVGGVGVGGVGVGGVGVGGVGLWLEHHHHHH

(19) United States
(12) Patent Application Publication
(10) Pub. No.: US 2026/0274924 A1
(43) Pub. Date: Sep. 17, 2026

(54) METHOD FOR PRODUCING POLYPEPTIDE

(71) Applicant: National University Corporation Tokai National Higher Education and Research System, Aichi (JP)

(72) Inventors: Shunsuke OISHI, Aichi (JP); Ayae NARUTAKI, Aichi (JP)

(73) Assignee: National University Corporation Tokai National Higher Education and Research System, Aichi (JP)

(21) Appl. No.: 19/469,090

(22) PCT Filed: Mar. 21, 2024
(86) PCT No.: PCT/JP2024/011171

§ 371 Date: Sep. 25, 2025

(30) Foreign Application Priority Data
Mar. 27, 2023 (JP) 2023-049791

(51) Int. Cl. C07K 14/78; C07K 1/107

(52) U.S. Cl. CPC C07K 14/78; C07K 1/1075

(57) ABSTRACT

Provided is a method for producing an elastin-mimetic polypeptide without relying on genetic engineering technology. The method for producing a polypeptide comprises an elastin-like block peptide sequence that comprises a G sequence block consisting of (SEQ ID NO: 1: X¹GGX²G)ₙ, wherein each X¹ is the same or different and represents V or L, each X² is the same or different and represents V or L, and n represents an integer of 2 or more, and a P sequence block consisting of (SEQ ID NO: 2: VPGX³G)ₘ, wherein each X³ is the same or different and represents any amino acid, and m represents an integer of 5 or more, the method comprising: (a) synthesizing the polypeptide and/or split fragments of the polypeptide by solid-phase synthesis; and/or (b) linking terminal regions of the multiple split fragments of the polypeptide by a chemical reaction.

### TECHNICAL FIELD

The present invention relates to, for example, a method for producing a polypeptide.

### BACKGROUND ART

Elastin is an important functional protein that imparts elasticity and stretchability to biological tissue; however, its use as a material is significantly delayed due to the difficulty of handling resulting from its high hydrophobicity. The present inventors have developed a double-hydrophobic elastin-mimetic polypeptide (GPG) that forms self-assembling nanofibers in water (Non-patent Literature (NPL) 1). The resulting fibers can form physical gels and can be expected to be applied to biomaterials (e.g., artificial blood vessels), cell culture substrates, and functional cosmetics.

### CITATION LIST

### Non-Patent Literature

NPL 1: Biomacromolecules. 14 (2013) 1028-1034. https://doi.org/10.1021/bm301887m.
NPL 2: Journal of Peptide Science. 2016 May; 22(5): 334-42.

### SUMMARY OF INVENTION

### Technical Problem

In NPL 1, GPG is produced intracellularly using genetic engineering technology. However, this production technique is not desirable due to biological impurities, difficulty in quality control, and consumer resistance to recombinant technology.

On the other hand, elastin-mimetic polypeptides have been considered difficult to synthesize chemically because they contain more than several tens of amino acid residues (NPL 2). Further, it has been considered that when multiple fragments are linked by a chemical reaction, linkage sites within the resulting elastin-mimetic polypeptide, which are formed by the above chemical reaction, inhibit the fiber formation of the elastin-mimetic polypeptide.

An object of the present invention is to provide a method for producing an elastin-mimetic polypeptide without relying on genetic engineering technology.

### Solution to Problem

The present inventors conducted extensive research to achieve the above object and found that the object can be achieved by a method for producing a polypeptide comprising an elastin-like block peptide sequence that comprises a G sequence block consisting of (SEQ ID NO: 1: X¹GGX²G)ₙ, wherein each X¹ is the same or different and represents V or L, each X² is the same or different and represents V or L, and n represents an integer of 2 or more, and a P sequence block consisting of (SEQ ID NO: 2: VPGX³G)ₘ, wherein each X³ is the same or different and represents any amino acid, and m represents an integer of 5 or more, the method comprising: (a) synthesizing the polypeptide and/or split fragments of the polypeptide by solid-phase synthesis; and/or (b) linking terminal regions of the multiple split fragments of the polypeptide by a chemical reaction. The inventors conducted further research on the basis of this finding and completed the present invention. Specifically, the present invention includes the following subject matter.

Item 1. A method for producing a polypeptide comprising an elastin-like block peptide sequence that comprises a G sequence block consisting of (SEQ ID NO: 1: X¹GGX²G)ₙ, wherein each X¹ is the same or different and represents V or L, each X² is the same or different and represents V or L, and n represents an integer of 2 or more, and a P sequence block consisting of (SEQ ID NO: 2: VPGX³G)ₘ, wherein each X³ is the same or different and represents any amino acid, and m represents an integer of 5 or more,
the method comprising:
(a) synthesizing the polypeptide and/or split fragments of the polypeptide by solid-phase synthesis; and/or
(b) linking terminal regions of the multiple split fragments of the polypeptide by a chemical reaction.

Item 2. The production method according to Item 1, wherein the sequence structure of the elastin-like block peptide sequence is, from the N-terminal side, G sequence block-P sequence block-G sequence block, G sequence block-P sequence block-P sequence block-G sequence block, P sequence block-G sequence block, or G sequence block-P sequence block.

Item 3. The production method according to Item 1 or 2, wherein the method comprises step (a), and the split fragments of the polypeptide each have 30 or more amino acid residues.

Item 4. The production method according to Item 3, wherein the split fragments of the polypeptide each have 50 or more amino acid residues.

Item 5. The production method according to any one of Items 1 to 4, wherein the method comprises step (a), and in step (a), the split fragments each contain all or part of the P sequence block.

Item 6. The production method according to any one of Items 1 to 5, wherein the method comprises step (a), and the polypeptide and/or each split fragment of the polypeptide contains a hydrophilic amino acid-rich sequence.

Item 7. The production method according to any one of Items 1 to 6, wherein the method comprises step (b), and the chemical reaction in step (b) is a reaction between a thiol group of an amino acid residue a in the terminal region of one split fragment a and a vinyl group or a maleimide group added to an amino acid residue b in the terminal region of the other split fragment b.

Item 8. The production method according to Item 7, wherein a group represented by formula (A):

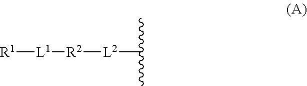

wherein R¹ represents a vinyl group or a maleimide group, L¹ represents a linker containing an amide bond or an ester bond, R² represents an aromatic ring, and L² represents a linker, is added to the terminal region of the split fragment b.

Item 9. The production method according to any one of Items 1 to 8, wherein the polypeptide has 80 or more amino acid residues.

Item 10. The production method according to any one of Items 1 to 8, wherein the polypeptide comprises an elastin-like block peptide sequence that comprises a G sequence block consisting of (SEQ ID NO: 1: X¹GGX²G)ₙ, wherein each X¹ is the same or different and represents V or L, each X² is the same or different and represents V or L, and n represents an integer of 2 or more, and a P sequence block consisting of (SEQ ID NO: 2: VPGX³G)ₘ, wherein each X³ is the same or different and represents any amino acid, and m represents an integer of 5 or more, and
(c) the sequence structure of the elastin-like block peptide sequence is G sequence block-P sequence block from the N-terminal side, and/or
(d) m is an integer of 5 to 22.

Item 11. A polypeptide comprising an elastin-like block peptide sequence that comprises a G sequence block consisting of (SEQ ID NO: 1: X¹GGX²G)ₙ, wherein each X¹ is the same or different and represents V or L, each X² is the same or different and represents V or L, and n represents an integer of 4 or more, and a P sequence block consisting of (SEQ ID NO: 2: VPGX³G)ₘ, wherein each X³ is the same or different and represents any amino acid, and m represents an integer of 5 or more, and
(c) the sequence structure of the elastin-like block peptide sequence is G sequence block-P sequence block from the N-terminal side, and/or
(d) m is an integer of 5 to 22.

Item 12. The polypeptide according to Item 11, wherein m is 13 to 22.

Item 13. A gel composition comprising the polypeptide according to Item 11 or 12.

### Advantageous Effects of Invention

The present invention provides a method for producing an elastin-mimetic polypeptide without relying on genetic engineering technology. The present invention also provides an elastin-mimetic polypeptide with a novel structure suitable for production by the method.

### BRIEF DESCRIPTION OF DRAWINGS

FIG. 1 shows the MALDI-TOF MS data and HPLC data of the polypeptide (MG5PPPPP) synthesized in Test Example 1.

FIG. 2 shows the MALDI-TOF MS data of the polypeptide (MG5P) synthesized in Test Example 2.

FIG. 3 shows the MALDI-TOF MS data of the polypeptide (MG5PP) synthesized in Test Example 2.

FIG. 4 shows the MALDI-TOF MS data of the polypeptide (PHis) synthesized in Test Example 2.

FIG. 5 shows the MALDI-TOF MS data of the polypeptide (PPHis) synthesized in Test Example 2.

FIG. 6 shows the MALDI-TOF MS data of the polypeptide (PPPHis) synthesized in Test Example 2.

FIG. 7 shows the HPLC data and MALDI-TOF MS data of the polypeptide (MG5P-PHis) synthesized in Test Example 3.

FIG. 8 shows the HPLC data and MALDI-TOF MS data of the polypeptide (MG5P-PPHis) synthesized in Test Example 3.

FIG. 9 shows the HPLC data and MALDI-TOF MS data of the polypeptide (MG5P-PPPHis) synthesized in Test Example 3.

FIG. 10 shows the HPLC data and MALDI-TOF MS data of the polypeptide (MG5PP-PPPHis) synthesized in Test Example 3.

FIG. 11 shows an atomic force microscope image of the MG5P-PPPHis gel of Test Example 4.

FIG. 12 shows the frequency dispersion measurement results of the storage modulus (G′) in Test Example 5. GP2 shows the measurement results of the MG5P-PHis gel, GP3 shows the measurement results of the MG5P-PPHis gel, GP4 shows the measurement results of the MG5P-PPPHis gel, and GP5 shows the measurement results of the MG5PP-PPPHis gel.

FIG. 13 shows the frequency dispersion measurement results of the loss tangent (tan δ) in Test Example 5. GP2 shows the measurement results of the MG5P-PHis gel, GP3 shows the measurement results of the MG5P-PPHis gel, GP4 shows the measurement results of the MG5P-PPPHis gel, and GP5 shows the measurement results of the MG5PP-PPPHis gel.

FIG. 14 shows the strain dispersion measurement results of the loss tangent (tan δ) in Test Example 5. GP2 shows the measurement results of the MG5P-PHis gel, GP3 shows the measurement results of the MG5P-PPHis gel, GP4 shows the measurement results of the MG5P-PPPHis gel, and GP5 shows the measurement results of the MG5PP-PPPHis gel.

FIG. 15 shows the MALDI-TOF MS data of the polypeptide (MPP) synthesized in Test Example 6.

FIG. 16 shows the MALDI-TOF MS data of the polypeptide (MPPP) synthesized in Test Example 6.

FIG. 17 shows the MALDI-TOF MS data of the polypeptide (MG4PP) synthesized in Test Example 6.

FIG. 18 shows the MALDI-TOF MS data of the polypeptide (PG5His) synthesized in Test Example 6.

FIG. 19 shows the MALDI-TOF MS data of the polypeptide (PPG4His) synthesized in Test Example 6.

FIG. 20 shows the MALDI-TOF MS data of the polypeptide (PPG5His) synthesized in Test Example 6.

FIG. 21 shows the HPLC data and MALDI-TOF MS data of the polypeptide (MPP-PG5His) synthesized in Test Example 7.

FIG. 22 shows the HPLC data and MALDI-TOF MS data of the polypeptide (MPP-PPG5His) synthesized in Test Example 7.

FIG. 23 shows the HPLC data and MALDI-TOF MS data of the polypeptide (MPPP-PPG5His) synthesized in Test Example 7.

FIG. 24 shows the HPLC data and MALDI-TOF MS data of the polypeptide (MPP-PPG4His) synthesized in Test Example 7.

FIG. 25 shows the HPLC data and MALDI-TOF MS data of the polypeptide (MG4PP-PPHis) synthesized in Test Example 7.

### DESCRIPTION OF EMBODIMENTS

In the present specification, the terms “comprise,” “contain,” and “include” include the concepts of “comprising,” “containing,” “consisting essentially of,” and “consisting of.”

In the present specification, amino acid residues in an amino acid sequence may be represented, for example, by amino acids or specific amino acid names (e.g., valine and leucine). Amino acid residues in an amino acid sequence may also be represented by the one-letter code of amino acids.

In an embodiment, the present invention relates to a method for producing a polypeptide comprising an elastin-like block peptide sequence that comprises a G sequence block consisting of (SEQ ID NO: 1: X¹GGX²G)ₙ, wherein each X¹ is the same or different and represents V or L, each X² is the same or different and represents V or L, and n represents an integer of 2 or more, and a P sequence block consisting of (SEQ ID NO: 2: VPGX³G)ₘ, wherein each X³ is the same or different and represents any amino acid, and m represents an integer of 5 or more (which may be referred to as “the polypeptide of the present invention” in the present specification), the method comprising: (a) synthesizing the polypeptide and/or split fragments of the polypeptide by solid-phase synthesis; and/or (b) linking terminal regions of the multiple split fragments of the polypeptide by a chemical reaction (which may be referred to as “the production method of the present invention” in the present specification). This is explained below.

The polypeptide of the present invention, which is the production target, is explained below.

The G sequence block is not particularly limited as long as it is a block consisting of a repeat sequence (X¹GGX²G)ₙ of the amino acid sequence (X¹GGX²G) set forth in SEQ ID NO: 1. The G sequence block is typically a block that can take a 3-sheet structure. The G sequence block can impart the ability to form fibers by self-assembly to the polypeptide.

Each X¹ is the same or different and represents V or L, and each X² is the same or different and represents V or L. X¹ and X² are preferably V.

n represents an integer of 2 or more. When n is 2 or more, the polypeptide of the present invention can form a fibrous self-assembly. n is preferably 3 or more, more preferably 4 or more, even more preferably 4 to 20, still even more preferably 4 to 12, especially more preferably 4 to 8, and especially even more preferably 4 to 6.

The P sequence block is not particularly limited as long as it is a block consisting of a repeat sequence (VPGX³G)ₘ of the amino acid sequence (VPGX³G) set forth in SEQ ID NO: 2. The P sequence block is typically a block that can take a S-turn structure. The P sequence block allows the polypeptide to have a lower critical solution temperature (LCST) and to self-assemble at the LCST or higher to phase separate from an aqueous solution. The presence of the P sequence block, or the P sequence block in combination with other sequence structures of the polypeptide of the present invention, further improves the efficiency of solid-phase synthesis.

The LCST of the polypeptide of the present invention (solvent: water, concentration: 0.03 wt %) is, for example, 10 to 30° C., and preferably 15 to 25° C.

Each X³ is the same or different and represents any amino acid. Examples of amino acids represented by X³ include amino acids having a basic side chain such as lysine, arginine, and histidine; amino acids having an acidic side chain such as aspartic acid and glutamic acid; amino acids having an uncharged polar side chain such as glycine, asparagine, glutamine, serine, threonine, tyrosine, and cysteine; amino acids having a nonpolar side chain such as alanine, valine, leucine, isoleucine, proline, phenylalanine, methionine, and tryptophan; amino acids having a β-branched side chain such as threonine, valine, and isoleucine; amino acids having an aromatic side chain such as tyrosine, phenylalanine, tryptophan, and histidine; and the like. The amino acids represented by X³ are preferably amino acids other than proline.

The LCST can be adjusted by the amino acids represented by X³. If the amino acids represented by X³ are hydrophilic amino acids, the LCST tends to become too high, which is not preferable. From the viewpoint of easily adjusting the LCST to an appropriate (physiologically acceptable) temperature (e.g., 30 to 40° C.), it is preferred that some of the amino acids represented by X³ in the P sequence block are non-aromatic, hydrophobic amino acids, such as valine and leucine (preferably valine), and the other amino acids are aromatic, hydrophobic amino acids, such as phenylalanine and tryptophan (preferably phenylalanine). In this case, the proportion of the number of non-aromatic, hydrophobic amino acids represented by X³ relative to the total number of X³s in the P sequence block is, for example, 60 to 95%, preferably 70 to 90%, and more preferably 75 to 85%, and the proportion of the number of aromatic, hydrophobic amino acids represented by X³ relative to the total number of X³s in the P sequence block is, for example, 5 to 40%, preferably 10 to 30%, and more preferably 15 to 25%. Further, in this case, the aromatic, hydrophobic amino acids represented by X³ preferably appear as dispersed as possible in the P sequence block. For example, it is preferred that a repeat unit in which X³ represents an aromatic, hydrophobic amino acid appears at a ratio of one to, for example, one to ten repeat units (VPGX³G), preferably two to eight repeat units (VPGX³G), more preferably three to seven repeat units (VPGX³G), even more preferably four to six repeat units (VPGX³G), and particularly preferably five repeat units (VPGX³G).

m represents an integer of 5 or more. From the viewpoint of the high storage modulus, low loss tangent, and ability to maintain more solid-like properties against strain of a gel obtained from the polypeptide of the present invention, m is preferably 5 to 25, more preferably 8 to 25, even more preferably 13 to 25, still even more preferably 13 to 22, especially more preferably 13 to 20, and particularly preferably 14 to 17. Further, in an embodiment of the present invention, m can be 5 to 22 or 13 to 22.

The elastin-like block peptide sequence is not particularly limited as long as it comprises the G sequence block and the P sequence block. The number of G sequence blocks in the elastin-like block peptide sequence is not particularly limited, and is, for example, 1 to 5, preferably 1 to 4, more preferably 1 to 3, even more preferably 1 or 2, and particularly preferably 1. The number of P sequence blocks in the elastin-like block peptide sequence is not particularly limited, and is, for example, 1 to 5, preferably 1 to 4, more preferably 1 to 3, even more preferably 1 or 2, and particularly preferably 1.

The blocks (the G sequence block and the P sequence block, the G sequence block and the G sequence block, and the P sequence block and the P sequence block) may be directly linked or may be linked via a linker sequence.

The linker sequence is not particularly limited as long as it does not significantly reduce the fiber-forming ability of the elastin-like block peptide sequence due to its self-assembly, and usually any amino acid or amino acid sequence can be used without significant limitation. The linker sequence is, for example, 1 to 20, preferably 1 to 15, more preferably 1 to 10, even more preferably 1 to 8, and still even more preferably 2 to 8 amino acids in length. Specific examples of the linker sequence include the amino acid sequence set forth in SEQ ID NO: 9: LWLGSG, the amino acid sequence set forth in KL, and the like. Furthermore, amino acid sequences in which one or more (for example, one to five, preferably one to three, more preferably one or two, and even more preferably one) amino acids are mutated (for example, substituted, deleted, inserted, and/or added, preferably substituted, more preferably conservatively substituted) relative to any of these amino acid sequences can also be used.

In the present specification, “conservative substitution” means the substitution of an amino acid with another amino acid having a similar side chain. For example, the substitution between amino acids having a basic side chain, such as lysine, arginine, or histidine, is considered to be a conservative substitution. The following substitutions between other amino acids are also considered to be a conservative substitution: the substitution between amino acids having an acidic side chain such as aspartic acid or glutamic acid; the substitution between amino acids having an uncharged polar side chain such as glycine, asparagine, glutamine, serine, threonine, tyrosine, or cysteine; the substitution between amino acids having a nonpolar side chain such as alanine, valine, leucine, isoleucine, proline, phenylalanine, methionine, or tryptophan; the substitution between amino acids having a β-branched side chain such as threonine, valine, or isoleucine; and the substitution between amino acids having an aromatic side chain such as tyrosine, phenylalanine, tryptophan, or histidine.

From the viewpoint of the ability to form fibers by self-assembly, the sequence structure of the elastin-like block peptide sequence is preferably, from the N-terminal side, G sequence block-P sequence block-G sequence block, G sequence block-P sequence block-P sequence block-G sequence block, P sequence block-G sequence block, or G sequence block-P sequence block. The sequence structure is particularly preferably G sequence block-P sequence block. In these structures, “-” indicates a direct link (between blocks) or another sequence (e.g., a linker sequence).

The polypeptide of the present invention preferably contains a hydrophilic amino acid-rich sequence. The hydrophilic amino acid-rich sequence can further improve the efficiency of solid-phase synthesis, ease of purification associated with improved polypeptide solubility, gel-forming properties, and the like. The hydrophilic amino acid-rich sequence is not particularly limited as long as it is a sequence containing a high proportion of hydrophilic amino acids (amino acids having a basic side chain, amino acids having an acidic side chain, and amino acids having an uncharged polar side chain). Examples include amino acid sequences having a length of, for example, 3 to 15 or 5 to 10 amino acids at a ratio of, for example, 50% or more, 60% or more, 70% or more, 80% or more, or 90% or more. The hydrophilic amino acid-rich sequence is preferably a histidine repeat sequence (His-tag sequence). Other amino acid sequences preferably contain amino acids having functional groups that can be used in the crosslinking reaction (e.g., lysine and cysteine).

When the polypeptide of the present invention contains a hydrophilic amino acid-rich sequence, the cell adhesive sequence is positioned at a terminus or the termini (the N-terminus and/or the C-terminus) of the polypeptide of the present invention, preferably at the C-terminus. The hydrophilic amino acid-rich sequence may be positioned at either the N-terminus or the C-terminus, or both.

The polypeptide of the present invention may be terminated with a cell adhesive sequence. The cell adhesive sequence is not particularly limited, and various sequences can be used. The cell adhesive sequence may be one that does not have cell selectivity, such as RGDS (SEQ ID NO: 10), or one that has cell selectivity (e.g., REDV (SEQ ID NO: 22)). Examples of sequences that have cell selectivity include HHH, VVV, TIT, TGA, NNN, KKK, AAA, RRR, YYY, TTT, GAT, GGG, PGH, GQA, QGD, GIG, EKG, KGK, QGF, GMK, GLS, CAG, CNG, KGT, PLG, NRG, CSG, LGL, AVG, GHP, GLI, GVG, GPS, SPG, GPP, GIS, GYL, GEK, QGE, CNY, FPG, GAP, APG, GEC, LPG, GPR, PCG, GDV, IGG, CDG, AVA, FLM, GFD, GTP, GPY, VSG, DGR, GIT, GFL, ASG, GCP, NQG, SGL, GGA, PDG, QAL, GLK, GSP, GEP, GNS, AKG, DGY, TGP, VGP, SLW, AAG, AGA, ARG, GRD, EGF, GSC, PGQ, HSQ, EAP, RGP, PGD, CNI, GFG, GPT, GDQ, KGE, PFI, QGP, SYW, LPGFPGLK (SEQ ID NO: 11), LPGFPGTP (SEQ ID NO: 12), GPPGLSGPP (SEQ ID NO: 13), FPGPPGPP (SEQ ID NO: 14), LPGLPGPP (SEQ ID NO: 15), FPGLPGPP (SEQ ID NO: 16), GPPGPPGSPG (SEQ ID NO: 17), LPGPPGPP (SEQ ID NO: 18), FPGSPGFPG (SEQ ID NO: 19), GSPGLPGTP (SEQ ID NO: 20), IGLSGEKG (SEQ ID NO: 21), and the like.

When the polypeptide of the present invention contains a cell adhesive sequence, the cell adhesive sequence is positioned at a terminus or the termini (the N-terminus and/or the C-terminus) of the polypeptide of the present invention, preferably at the C-terminus. The cell adhesive sequence may be positioned at either the N-terminus or the C-terminus, or both. In a preferred embodiment of the present invention, the cell adhesive sequence is positioned at only one terminus.

The number of amino acid residues in the elastin-like block peptide sequence is preferably 50% or more, more preferably 60% or more, even more preferably 70% or more, still even more preferably 80% or more, and particularly preferably 90% or more, based on the number of amino acid residues in the polypeptide of the present invention, which is taken as 100%.

The number of amino acid residues in the polypeptide of the present invention is, for example, 80 or more, preferably 80 to 250, more preferably 90 to 200, and even more preferably 100 to 180.

The polypeptide of the present invention can be a polymer consisting of amino acids linked by amide bonds (a typical polypeptide), or a polymer in which the terminal amino acid residues of the typical polypeptide are linked by a chemical reaction (a linked polypeptide). In the latter case, linkers (the number of main chain atoms is, for example, 2 to 25, 5 to 20, or 7 to 15) containing bonds by a chemical reaction are placed between multiple (e.g., 2 to 5, 2 to 4, 2 or 3, or 2) typical polypeptides. The main chain is a chain that connects the terminal amino acid residues of the typical polypeptide, and when a ring is present, it is a chain that traces the atoms in the ring so that the chain length is the shortest.

The polypeptide of the present invention may be chemically modified as long as the gel-forming properties are not significantly impaired. The presence or absence of gel-forming properties can be evaluated according to Test Examples 4 and 5 described later.

The polypeptide of the present invention may have a carboxyl group (—COOH), carboxylate (—CO—), amide (—CONH₂), or ester (—COOR) at the C-terminus.

“R” in the ester is, for example, a C₁₋₆ alkyl group such as methyl, ethyl, n-propyl, isopropyl, or n-butyl; a C₃₋₈ cycloalkyl group such as cyclopentyl or cyclohexyl; a C₆₋₁₂ aryl group such as phenyl or α-naphthyl; a phenyl-C₁₋₂ alkyl group such as benzyl or phenethyl; a C₇₋₁₄ aralkyl group such as an α-naphthyl-C₁₋₂ alkyl group such as α-naphthyl methyl; or a pivaloyloxymethyl group.

The polypeptide of the present invention may have an amidated or esterified carboxyl group (or carboxylate), which is not the carboxyl group at the C-terminus. The ester in this case may be, for example, the ester at the C-terminus described above.

The polypeptide of the present invention further encompasses polypeptides having the amino group of the N-terminal amino acid residue protected by a protective group (e.g., a C₁₋₆ acyl group including C₁₋₆ alkanoyl such as a formyl group and an acetyl group); polypeptides having the N-terminal glutamine residue pyroglutamated that can be formed due to cleavage in vivo; polypeptides having a substituent (e.g., —OH, —SH, an amino group, an imidazole group, an indole group, and a guanidino group) on a side chain of an amino acid in the molecule protected by an appropriate protective group (e.g., a C₁₋₆ acyl group including a C₁₋₆ alkanoyl group such as a formyl group and an acetyl group); conjugated proteins such as glycoproteins having a sugar chain bonded thereto; and the like.

The polypeptide of the present invention may be in the form of a pharmaceutically acceptable salt formed with an acid or base. The salt can be any pharmaceutically acceptable salt, and can be either an acid salt or a basic salt. Examples of acid salts include inorganic acid salts, such as hydrochloride, hydrobromide, sulfate, nitrate, and phosphate; organic acid salts, such as acetate, propionate, tartrate, fumarate, maleate, malate, citrate, methanesulfonate, and para-toluenesulfonate; amino acid salts, such as aspartate and glutamate; and the like. Examples of basic salts include alkali metal salts, such as sodium salts and potassium salts; alkaline-earth metal salts, such as calcium salts and magnesium salts; and the like.

The polypeptide of the present invention may be in the form of a solvate. The solvent can be any pharmaceutically acceptable solvent, and may be, for example, water, ethanol, glycerol, or acetic acid.

In step (a), the polypeptide of the present invention and/or split fragments of the polypeptide of the present invention are synthesized by solid-phase synthesis.

The split fragments of the polypeptide of the present invention are polypeptides consisting of a part of the amino acid sequence of the polypeptide of the present invention, and are not particularly limited in that respect.

The number of amino acid residues in each split fragment is, for example, 30 or more, preferably 40 or more, and more preferably 50 or more. Even with such long-chain polypeptides, as long as they are split fragments of the polypeptide of the present invention, solid-phase synthesis is possible.

The split fragment preferably contains all or part of the P sequence block of the polypeptide of the present invention. This can further improve the efficiency of solid-phase synthesis. The number of repeats of the P sequence block (SEQ ID NO: 2: VPGX³G) within the split fragment may be preferably 3 or more, and more preferably 5 or more. The upper limit thereof is not particularly limited and can be the upper limit of the number of repeats of the P sequence block in the polypeptide of the present invention. The number of amino acid residues in the P sequence block is preferably 25% or more, more preferably 30% or more, even more preferably 35% or more, still even more preferably 40% or more, especially preferably 50% or more, especially more preferably 60% or more, and particularly preferably 70% or more, based on the number of amino acid residues in the split fragment, which is taken as 100%.

In an embodiment of the present invention, the split fragment preferably does not contain the G sequence block of the polypeptide of the present invention. This can further improve the efficiency of solid-phase synthesis.

In an embodiment of the present invention, the split fragment preferably contains the hydrophilic amino acid-rich sequence of the polypeptide of the present invention. This can further improve the efficiency of solid-phase synthesis, ease of purification associated with improved polypeptide solubility, gel-forming properties, and the like.

In an embodiment of the present invention, for use in the linkage by a chemical reaction in step (b) described later, the amino acid residues in the terminal region of the split fragment can be amino acid residues having a side chain that can be used in the chemical reaction, or amino acid residues to which groups that can be used in the chemical reaction have been added.

Examples of the amino acid residues in the terminal region include amino acid residues in the range of 1 to 5 amino acids from the terminal, preferably amino acid residues in the range of 1 to 3 amino acids, more preferably amino acid residues in the range of 1 to 2 amino acids, and particularly preferably the terminal amino acid residue.

The amino acid residue having a side chain that can be used in the chemical reaction is preferably a cysteine residue. The thiol group on the cysteine residue side chain can be used for linkage by a thiol-ene reaction or a Michael addition reaction.

Preferred examples of groups that can be used in the chemical reaction include a vinyl group and a maleimide group, with a vinyl group being particularly preferred. The vinyl group can be used in the thiol-ene reaction with the thiol group, and the maleimide group can be used in the Michael addition reaction with the thiol group. From the viewpoint of reaction efficiency in step (b) described later, a group represented by formula (A):

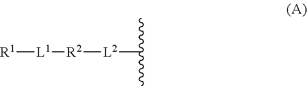

wherein R¹ represents a vinyl group or a maleimide group, L¹ represents a linker containing an amide bond (—C(═O)—NH—) or an ester bond (—C(═O)—O—), R² represents an aromatic ring, and L² represents a linker, is preferably added to the terminal region of the split fragment.

The maleimide group represented by R¹ is a group represented by formula (B):

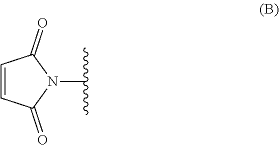 [SMILES: *N1C(=O)C=CC1=O]

The number of atoms constituting the main chain of the linker represented by L¹ is preferably 2 to 5, more preferably 2 to 4, even more preferably 2 or 3, and particularly preferably 2. The linker represented by L¹ has a chain structure that does not contain a ring structure. The linker represented by L¹ is particularly preferably an amide bond (—C(═O)—NH—).

The aromatic ring represented by R² can be, for example, an aromatic ring with 5 to 20 carbon atoms, preferably 5 to 12 carbon atoms, and more preferably 5 to 7 carbon atoms. The aromatic ring is particularly preferably a benzene ring.

The number of atoms constituting the main chain of the linker represented by L² is preferably 1 to 5, more preferably 1 to 3, and even more preferably 2. The linker represented by L² has a chain structure that does not contain a ring structure. The linker represented by L² is particularly preferably —CH₂—C(═O)—.

Solid-phase synthesis is not particularly limited and can be carried out according to or based on a known method.

The general steps of the solid-phase synthesis method are as follows. An amino acid whose N-terminus is protected by a protective group (typically Fmoc or Boc) (protected amino acid) is bound to a solid insoluble carrier (typically polystyrene resin beads) at the C-terminus of the protected amino acid, optionally through a linker. Next, unreacted protected amino acids, i.e., protected amino acids that are not bound to the carrier, are removed. Then, the protective group of the protected amino acid is removed under conditions that do not cause the protected amino acid bound to the carrier to be released from the carrier. Separately, a second protected amino acid is prepared by protecting the N-terminus of an amino acid to be bound to the carrier-bound amino acid by a protective group. The second protected amino acid is added to the amino acid bound to the carrier, and the N-terminus of the amino acid bound to the carrier is condensed with the C-terminus of the second protected amino acid. Next, unreacted second protected amino acids are removed, and then the protective group of the second protected amino acid is removed. A third amino acid whose N-terminus is protected by a protective group is added, and the N-terminus of the second amino acid is condensed with the C-terminus of the third protected amino acid. This procedure is repeated to synthesize a peptide having the desired amino acid sequence bound to the carrier. The peptide is separated from the carrier to obtain the desired peptide.

Examples of the protective group for the amino group on the main chain include 9-fluorenylmethyloxycarbonyl (Fmoc), tertiary-butoxycarbonyl (Boc), benzyloxycarbonyl (Z), tertiary-amyloxycarbonyl, isobornyloxycarbonyl, 4-methoxybenzyloxycarbonyl, 2-chlorobenzyloxycarbonyl (Cl—Z), 2-bromobenzyloxycarbonyl (Br—Z), adamantyloxycarbonyl, trifluoroacetyl, phthalyl, formyl, 2-nitrophenylsulfenyl, diphenylphosphinothioyl, and the like. Of these, from the viewpoint of peptide synthesis efficiency etc., F-moc and Boc are preferred, and Fmoc is particularly preferred.

If the amino acid has other groups (reactive side chains) that are not desired to react under the condensation reaction conditions, a protected amino acid in which these reactive side chains are protected by appropriate protective groups can be used. Examples of protective groups for carboxyl groups include alkyl esters (e.g., methyl, ethyl, propyl, butyl, tertiary butyl, cyclopentyl, cyclohexyl, cycloheptyl, cyclooctyl, 2-adamantyl, and other ester groups), benzyl esters, 2-nitrobenzyl esters, 4-methoxybenzyl esters, 4-chlorobenzyl esters, benzhydryl esters, phenacyl esters, benzyloxycarbonyl hydrazide, tert-butoxycarbonyl hydrazide, trityl hydrazide, and the like. The hydroxyl group of serine can be protected, for example, by esterification or etherification. Examples of groups suitable for esterification include lower alkanoyl groups such as acetyl (preferably alkanoyl groups having 1 to 3 carbon atoms), aroyl groups such as benzoyl, and groups derived from carbonic acid such as benzyloxycarbonyl and ethoxycarbonyl. Examples of groups suitable for etherification include benzyl, tetrahydropyranyl, and tertiary butyl groups. Examples of the protective group for the phenolic hydroxyl group of tyrosine include benzyl (Bzl), 2,6-dichlorobenzyl (Cl₂-Bzl), 2-nitrobenzyl, Br—Z, and tertiary butyl (tBu). Examples of the protective group for the imidazole of histidine include para-toluenesulfonyl (Tos), 4-methoxy-2,3,6-trimethylbenzenesulfonyl, dinitrophenyl (DNP), benzyloxymethyl, tert-butoxymethyl (Bum), Boc, trityl (Trt), Fmoc, and the like.

Examples of carriers include, but are not limited to, styrene resin, acrylamide resin, polyethylene glycol-acrylamide composite resin, polyoxyethylene grafted styrene resin, and the like. These may be crosslinked with divinylbenzene, and may be used singly or in combination of two or more. A wide range of known linkers can be used. Examples of linkers include, but are not limited to, chloromethyl, hydroxymethyl, benzhydrylamine, aminomethyl, 4-benzyloxybenzyl alcohol, 4-methylbenzhydrylamine, phenylacetamidomethyl, 4-hydroxymethylphenylacetamidomethyl, 4-(2′,4′-dimethoxyphenyl-hydroxymethyl)phenoxy, 2-chlorotrityl chloride resin, 4-hydroxymethylphenoxyacetic acid (HMPA), 4-hydroxymethylbenzoic acid (HMBA), 2,4-dimethoxy-4-hydroxybenzophenone, and the like.

A protective group remover may be selected appropriately, taking into consideration the protective group, solid-phase carrier, linker, etc. used. For example, a N,N-dimethylformamide (DMF) solution containing 20% v/v piperidine can be used to remove Fmoc. From the viewpoint of reducing environmental impact, to reduce the amount of organic solvent used in this step, an aqueous protective group remover, such as aqueous ethanol containing sodium hydroxide (e.g., 90% aqueous ethanol containing 0.1M NaOH), can be used. Other protective group removers include a 25% v/v hydrogen bromide-acetic acid solution, anhydrous hydrogen fluoride, methanesulfonic acid, trifluoromethanesulfonic acid, trifluoroacetic acid (TFA), and the like. In addition, a 2,4-dinitrophenyl group, which is used as the protective group for imidazole of histidine, can be removed by treatment with thiophenol, and a formyl group, which is used as the protective group for indole of tryptophan, can be removed by deprotection by acid treatment in the presence of 1,2-ethanedithiol, 1,4-butanedithiol, or the like, as mentioned above, or by alkaline treatment with dilute sodium hydroxide, dilute ammonia, or the like. In addition, the protection of functional groups that should not be involved in the condensation reaction, their protective groups, the removal of the protective groups, the activation of functional groups that are involved in the reaction, the reaction conditions, and the like can also be appropriately selected from known groups or known means.

The isolated peptide is purified as necessary by a conventional method, such as extraction, partition, reprecipitation, recrystallization, column chromatography, or high-performance liquid chromatography.

In step (b), the terminal regions of the multiple split fragments of the polypeptide of the present invention are linked together by a chemical reaction.

The number of split fragments linked in step (b) is not particularly limited, but can be, for example, 2 to 5, 2 to 4, 2 or 3, or 2. When the number of split fragments is 3 or more, the 3 or more split fragments may be linked simultaneously, or the 3 or more split fragments may be linked sequentially (2 split fragments are linked, and the resulting fragment is linked with another split fragment).

The chemical reaction in step (b) is not particularly limited as long as it is a reaction that can link two molecules by a covalent bond; however, from the viewpoints of reaction efficiency, regioselectivity, and the like, the chemical reaction is preferably a click reaction. Among click reactions, from the viewpoint of the safety of components used in the reaction for living organisms, for example, thiol-ene reactions, Michael addition reactions, and Huisgen cycloaddition reactions can be used, with thiol-ene reactions and Michael addition reactions being preferred, and thiol-ene reactions being particularly preferred.

The chemical reaction in step (b) is particularly preferably a reaction between a thiol group in an amino acid residue a in the terminal region of one fragment a and a vinyl group or maleimide group added to an amino acid residue b in the terminal region of the other fragment b. In this embodiment, from the viewpoint of reaction efficiency in step (b), it is preferred that the group represented by formula (A) described above is added to the terminal region of the split fragment b. It is also preferred that the amino acid residue a is an amino acid residue in the C-terminal region, while the amino acid residue b is an amino acid residue in the N-terminal region.

In step (b), the amount of one split fragment to be linked per mol of the other split fragment is, for example, 0.5 to 3 mol, and preferably 0.8 to 2 mol.

The solvent in step (b) is not particularly limited as long as it is a solvent that dissolves polypeptides; however, the solvent is particularly preferably water.

The reaction liquid in step (b) preferably contains a buffer, such as sodium carbonate buffer. The pH of the reaction liquid is preferably 8 to 12, more preferably 9 to 11, and particularly preferably 9.5 to 10.5.

The reaction liquid may contain additives, such as catalysts and polymerization initiators, as necessary, depending on the type of chemical reaction. During the reaction, radicals can be generated by light irradiation or heating, which can accelerate the reaction.

The reaction can be carried out under heating, at room temperature, or under cooling, and preferably at a temperature of 30 to 80° C., and more preferably 40 to 60° C. The reaction time is not particularly limited and can generally be 30 minutes to 30 hours.

After completion of the reaction, the solvent is distilled off, and the product is purified as necessary by a conventional method, such as extraction, partition, reprecipitation, recrystallization, column chromatography, or high-performance liquid chromatography.

Step (b) is a convergent synthetic route, and is therefore efficient and preferred. In addition, the purification of the split fragments, which are synthetic intermediates, can facilitate the purification procedure of the polypeptide of the present invention, which is the target product.

Step (b) can be used for combinatorial synthesis. That is, by preparing multiple types of split fragments in advance, these split fragments can be combined in any desired manner and subjected to step (b) to easily produce various types of the polypeptide of the present invention. The use of the library of the polypeptides of the present invention thus obtained makes it possible to more easily and efficiently screen for the polypeptide of the present invention that is optimal for any given purpose.

By using step (a) and/or step (b) (particularly preferably steps (a) and (b), from the viewpoints of production efficiency, safety for living organisms, and the like), elastin-mimetic polypeptides can be produced without relying on genetic engineering technology. The polypeptide of the present invention obtained by the production method of the present invention can maintain and/or exhibit the gelling properties of the polypeptide.

The production method of the present invention is suitable for the production of an elastin-mimetic polypeptide with a novel structure (novel polypeptide) comprising an elastin-like block peptide sequence that comprises a G sequence block consisting of (SEQ ID NO: 1: X¹GGX²G)ₙ, wherein each X¹ is the same or different and represents V or L, each X² is the same or different and represents V or L, and n represents an integer of 4 or more, and a P sequence block consisting of (SEQ ID NO: 2: VPGX³G)ₘ, wherein each X³ is the same or different and represents any amino acid, and m represents an integer of 5 or more, and
(c) the sequence structure of the elastin-like block peptide sequence is G sequence block-P sequence block from the N-terminal side, and/or
(d) m is an integer of 5 to 22.

The novel polypeptide is superior to conventional elastin-mimetic polypeptides in that the resulting gel has a high storage modulus and a low loss tangent, and is able to maintain more solid-like properties against strain. From these viewpoints, it is preferable to satisfy the above requirement (d), and it is particularly preferable to satisfy both the above requirements (c) and (d). From the same points of view, m is more preferably 8 to 22, even more preferably 13 to 22, still even more preferably 13 to 17, and particularly preferably 14 to 16.

A gel composition that comprises the polypeptide of the present invention can be obtained by dissolving the polypeptide of the present invention in a solvent at a temperature lower than the LCST (e.g., 0 to 15° C., 2 to 10° C.), heating the resulting solution to the LCST or higher (e.g., 30 to 50° C., 30 to 45° C., 30 to 40° C.), and allowing the solution to stand for a certain period of time (e.g., 1 to 300 hours, preferably 8 to 120 hours, more preferably 12 to 80 hours). As the solvent, water is used. A mixed solvent of water and an organic solvent can also be used.

From the viewpoint of gel-forming properties, the concentration of the polypeptide of the present invention in the gel composition of the present invention is, for example, 0.2 w/v % or more, preferably 0.3 w/v % or more, and more preferably 0.35 w/v % or more. From the viewpoint of easily allowing other components (e.g., components necessary or suitable for cell culture) to permeate the gel composition of the present invention, the concentration is, for example, 3.0 w/v % or less, preferably 2.0 w/v % or less, more preferably 1.5 w/v % or less, even more preferably 1.0 w/v % or less, and still even more preferably 0.8 w/v % or less.

### EXAMPLES

The present invention is described below in detail with reference to Examples; however, the present invention is not limited to these Examples.

### Test Example 1: Synthesis 1 of Polypeptide (Solid-Phase Synthesis Method)

The polypeptide with the following amino acid sequence and structure was synthesized by a solid-phase synthesis method. Amino acid residues are represented by a one-letter code.

### MG5PPPPP (SEQ ID NO: 3)

MKGSG(VGGVG)₅KL[(VPGVG)₂(VPGFG)(VPGVG)₂]₅LWLEHHHHHH

The details of the synthesis method are as follows.

### 1-1: Abbreviations

DMF: N,N-dimethylformamide
HATU: O-(7-azabenzotriazol-1-yl)-N,N,N′,N′-tetramethyluronium hexafluorophosphate
HCTU: O-(1H-6-chlorobenzotriazol-1-yl)-N,N,N′,N′-tetramethyluronium hexafluorophosphate
HPLC: high-performance liquid chromatography

### 1-2: Synthesis Reagents and Solvents

Fmoc-amino acid, HCTU, and HATU were purchased from Merck (Germany). HPLC-grade acetonitrile used for analysis and purification was purchased from Kanto Chemical Co., Inc. (Tokyo, Japan) and FUJIFILM Wako Pure Chemical Corporation (Japan, Osaka). DMF from FUJIFILM Wako Pure Chemical was used as is for solid-phase peptide synthesis. Other commercially available reagents were purchased from Merck (Germany), Kanto Chemical Co., Inc. (Tokyo, Japan), FUJIFILM Wako Pure Chemical Corporation (Osaka, Japan), and Tokyo Chemical Industry Co., Ltd. (Tokyo, Japan), and used without purification.

### 1-3: Peptide Solid-Phase Synthesis

The solid-phase synthesis of peptides was carried out either manually or using an automated synthesizer by the Fmoc method. The amino acid reagents used were Fmoc-Cys(Trt)-OH, Fmoc-Glu(OtBu)-OH, Fmoc-Gly-OH, Fmoc-His(1-Trt)-OH, Fmoc-Leu-OH, Fmoc-Lys(Boc)-OH, Fmoc-Phe-OH, Fmoc-Pro-OH, Fmoc-Ser(tBu)-OH, Fmoc-Trp(Boc)-OH, and Fmoc-Val-OH. The solid phase used was 2-chlorotriethyl polystyrene resin, and the first amino acid residue was manually loaded onto the resin. Thereafter, peptide elongation was performed according to the following standard protocol. Fmoc-deprotection was performed with 20% piperidine in DMF solution (8 minutes×2). Coupling was carried out in DMF for 60 minutes using Fmoc-amino acid (4 molar equivalents relative to the amount of amino acid loaded on the resin), HCTU or HATU (4 molar equivalents), and N-methylmorpholine (8 molar equivalents). Double coupling (repeated coupling steps) was carried out as necessary. After coupling, the unreacted free amino groups were capped by treatment with 20% acetic anhydride and 10% N-methylmorpholine in DMF for 10 minutes. Epimerization-prone amino acid residues were coupled using 1-hydroxybenzotriazole ester previously prepared with N,N′-diisopropylcarbodiimide.

### 1-4: Automated Peptide Synthesizers

The automated peptide synthesizers used were CS Bio 136X (CSBio) or Syro I (Biotage).

### 1-5: Manual Peptide Synthesis

Each monomer (1 molar equivalent) was dissolved in a minimum amount of DMF, and HATU (1 molar equivalent) and N-methylmorpholine (2 molar equivalents) were added to prepare a reaction solution. After pre-activation for 2 minutes, the solution was added to the resin in a reaction column equipped with a filter (Kokusan Chemical Co., Ltd., Tokyo), and the reaction was carried out at room temperature for 2 hours. The coupling was repeated as necessary to complete the reaction.

### 1-6: Cleavage from Resin and Deprotection

A cleavage solution (triisopropylsilane:water:TFA=1:2:98 v/v solution) was added to the obtained resin and shaken at room temperature for 2 hours. After the reaction, the resin was removed by filtration, and the filtrate was concentrated under reduced pressure. Ice-cold diethyl ether was added to the resulting solid, which was then vortexed and centrifuged, and the supernatant was removed. This washing with diethyl ether was repeated twice to obtain a crude product. The crude product was dissolved in acetonitrile aqueous solution containing 0.1 v/v % TFA, filtered through a 0.45 μm membrane filter, and then purified by preparative HPLC. The obtained fraction was lyophilized to obtain the target peptide.

### 1-7: Analysis and Purification by HPLC

Analysis and purification of the peptide and protein segments were performed using a JASCO HPLC system (Tokyo, Japan) or a Gilson HPLC system (USA) equipped with a dual pump, a mixer, an in-line degasser, and a tunable UV detector. The mobile phase was observed for absorption at wavelengths of 220 nm, 254 nm, and 301 nm. If necessary, the column was heated using a column heater or a water bath. The mobile phase used was acetonitrile aqueous solution containing 0.1 v/v % TFA.

Before sample injection, the column was usually pre-equilibrated for 10 minutes with the initial solvent composition. After sample injection, the column was run for 10 minutes with the initial solvent composition, and then a gradient run was performed to the final solvent composition (e.g., 50% acetonitrile). After the final solvent composition was reached, the solvent composition was changed to 95% acetonitrile within 1 minute, and the column was washed for 10 minutes. The solvent composition was further changed to 10% acetonitrile within 1 minute, and the procedure was completed.

### 1-8: Analytical HPLC

Analytical HPLC was performed using Capcell Pak MG-II (Shiseido Co., Ltd., 5 μm, pore diameter 120 Å, inner diameter 4.6 mm×250 mm), Jupiter C18 (Phenomenex, 5 μm, pore diameter 300 Å, inner diameter 4.6 mm×250 mm), or Jupiter C4 (Phenomenex, 5 μm, 300 Å, inner diameter 4.6 mm×250 mm) at a flow rate of 1 mL/min.

### 1-9: Preparative HPLC

Preparative HPLC was performed using Capcell Pak C18 UG80 (Shiseido Co., Ltd., 5 μm, pore diameter: 80 Å, inner diameter: 50 mm×250 m), Jupiter C18 (Phenomenex, 5 μm, pore diameter: 300 Å, inner diameter: 30 mm×250 mm), Jupiter C18 (Phenomenex, 5 μm, pore diameter: 300 Å, inner diameter: 21.2 mm×250 m), Jupiter C4 (Phenomenex, 5 μm, pore diameter 300 Å, inner diameter: 30 mm×250 mm), or Jupiter C4 (Phenomenex, 5 μm, pore diameter: 300 Å, inner diameter: 21.2 mm×250 mm).

### 1-10: Mass Spectrometry

Mass spectrometry of the compound was measured using a Microflex MALDI-TOF spectrometer (Bruker, Germany) with α-cyano-4-hydroxycinnamic acid (CHCA) as the matrix.

### Results

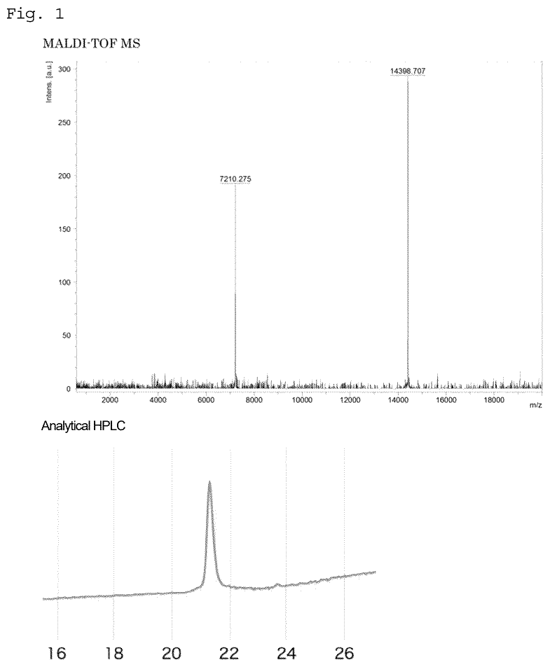

Mass spectrometry confirmed that the target polypeptide (MG5PPPPP) was synthesized (FIG. 1). The yield was 1.2%.

### Test Example 2: Synthesis 2 of Polypeptide (Solid-Phase Synthesis Method)

Polypeptides with the following amino acid sequences and structures were synthesized by a solid-phase synthesis method in the same manner as in Test Example 1. Only the C-terminal cysteine residues in MG5P and MG5PP are represented by a three-letter code, and the other amino acid residues are represented by a one-letter code. In Phis, PPHis, and PPPHis, —NH— on the left side of the N-terminal V (valine residue) is a group derived from the valine residue.

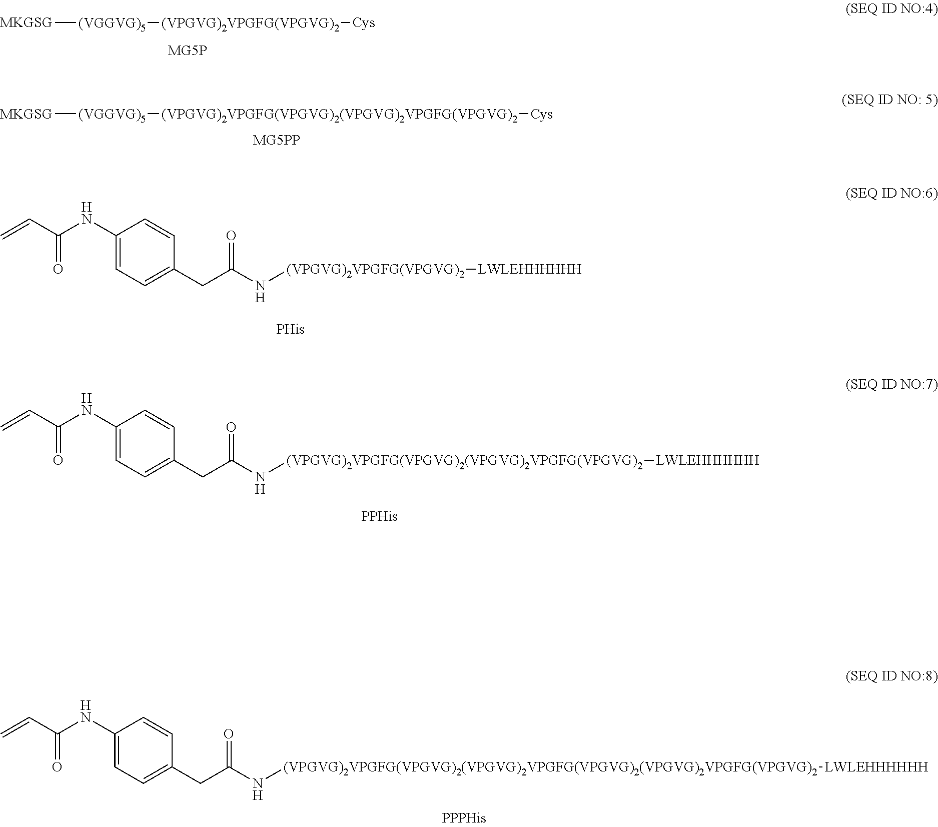

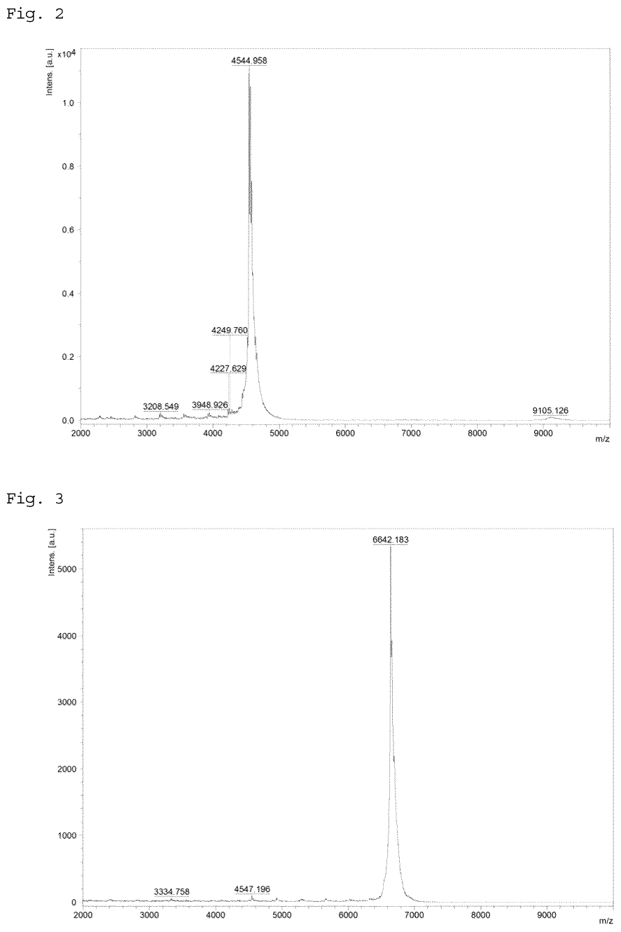

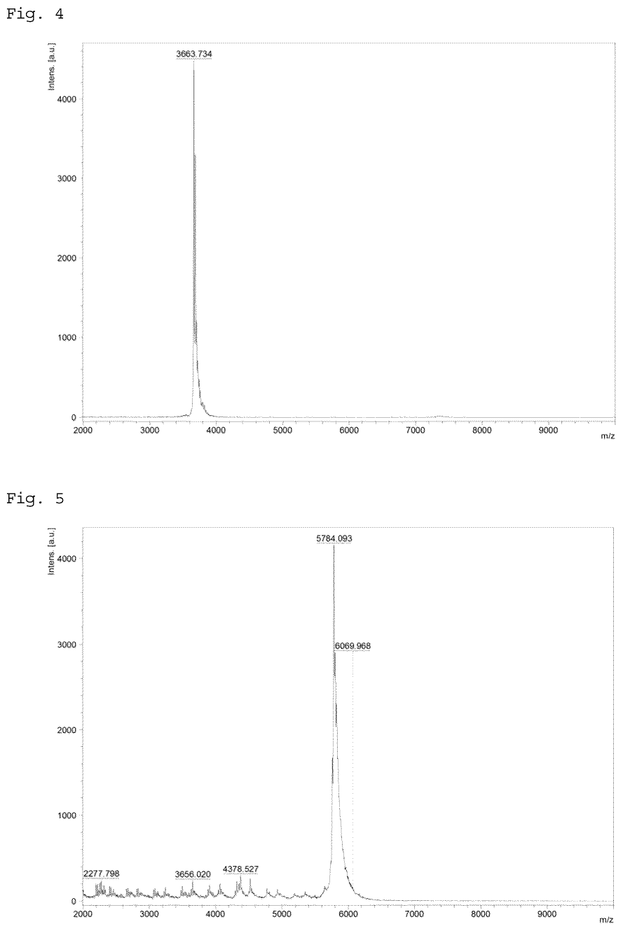

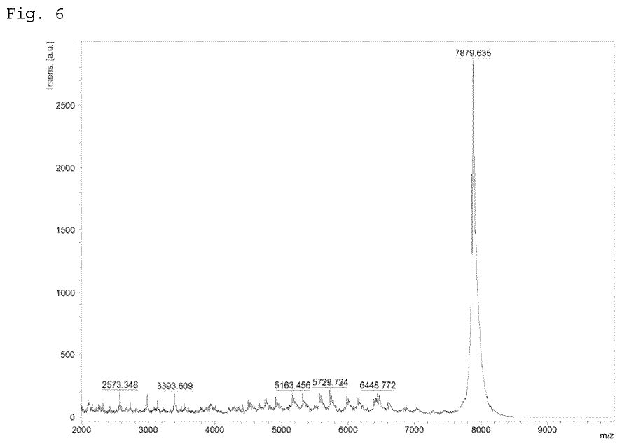

Mass spectrometry confirmed that the target polypeptides (MG5P, MG5PP, Phis, PPHis, and PPPHis) were synthesized (FIGS. 2 to 6). The yield was as follows.
MG5P: yield 19%
MG5PP: yield 16%
Phis: yield 27%
PPHis: yield 15%
PPPHis: yield 6%

### Test Example 3: Synthesis 3 of Polypeptide (Linkage by Chemical Reaction)

Polypeptides with the following amino acid sequences and structures were synthesized.

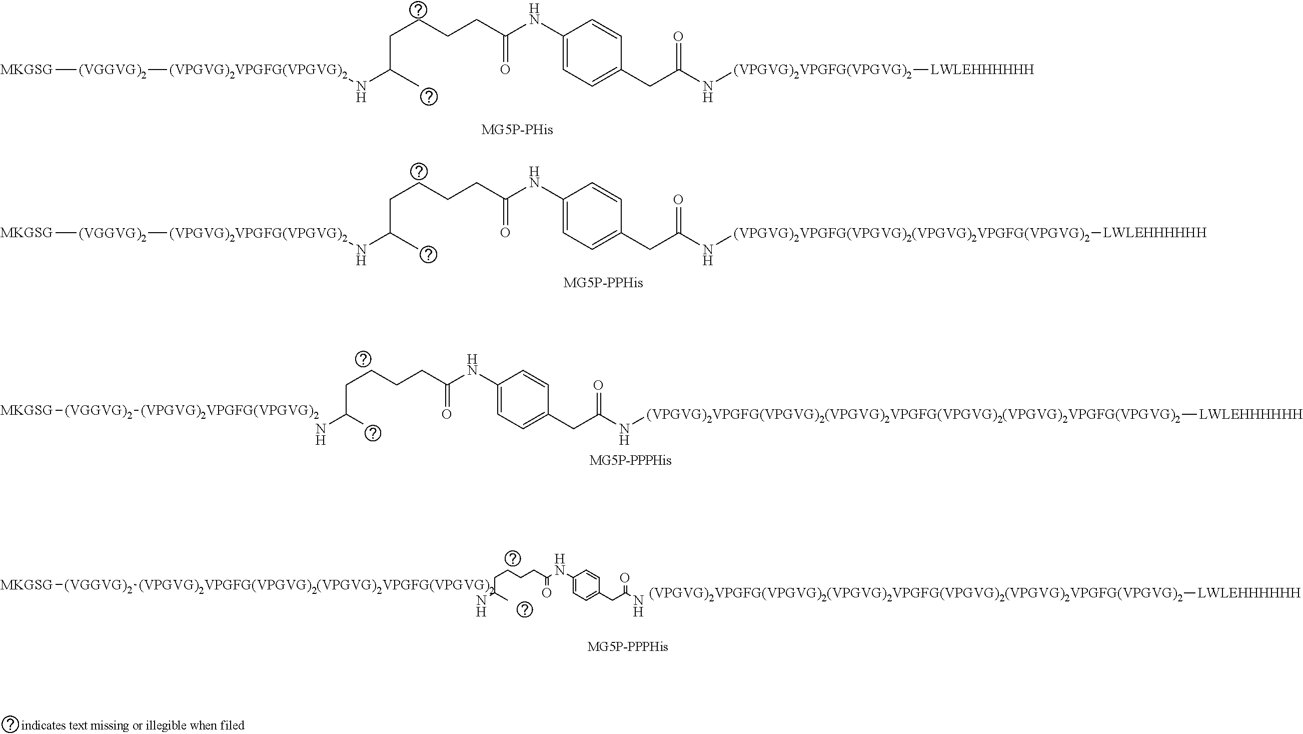

MG5P-PHis was synthesized by linking MG5P and PHis via the thiol-ene reaction. MG5P-PPHis was synthesized by linking MG5P and PPHis via the thiol-ene reaction. MG5P-PPPHis was synthesized by linking MG5P and PPPHis via the thiol-ene reaction. MG5PP-PPPHis was synthesized by linking MG5PP and PPPHis via the thiol-ene reaction.

The thiol-ene reaction was performed as follows. 1.5 molar equivalents of peptide segment 1 (MG5P or MG5PP) and 1.0 molar equivalent of peptide segment 2 (PHis, PPHis, or PPPHis) were weighed into a vial bottle, and sodium carbonate buffer (pH: 10) was added to adjust the concentration of peptide segment 2 to 10 mM. Dissolved oxygen in the buffer was removed in advance by bubbling nitrogen gas. The reaction solution was heated to 50° C. with stirring and reacted for 18 hours. The reaction was monitored by analytical HPLC. The mixture was then diluted with 50% acetonitrile aqueous solution containing 0.1 v/v % TFA and purified by preparative HPLC.

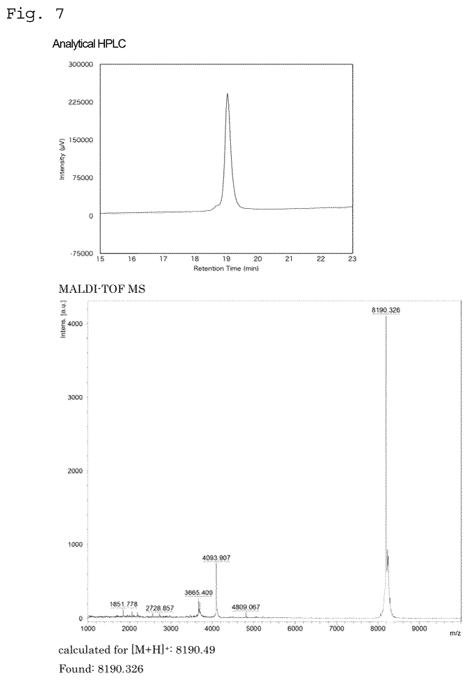

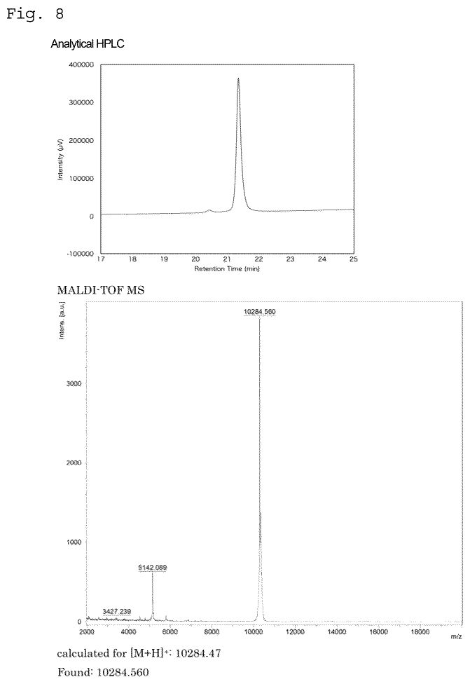

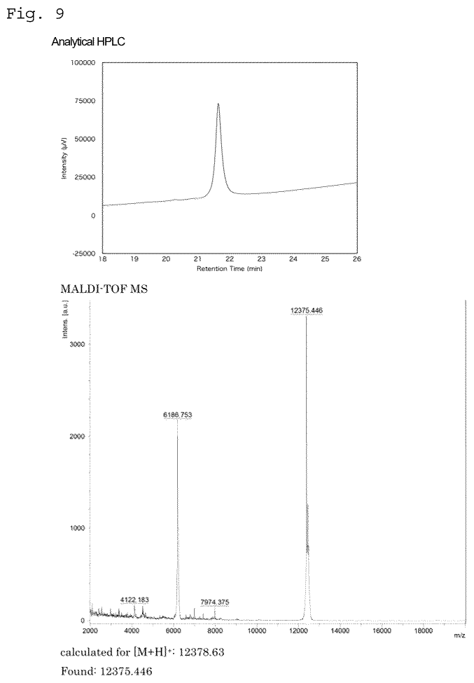

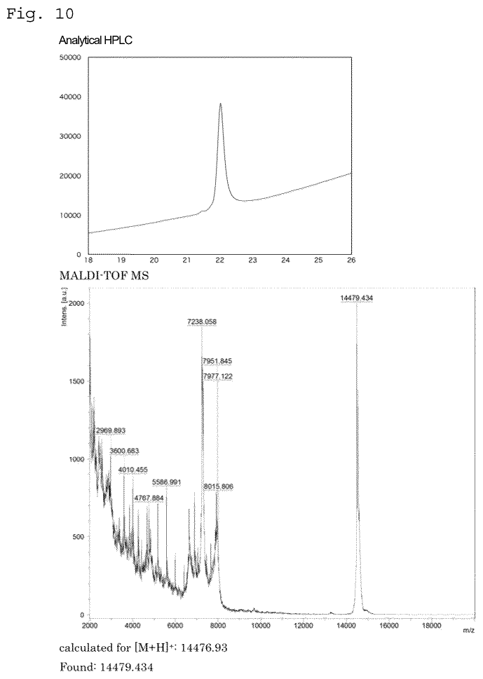

Mass spectrometry confirmed that the target polypeptides (MG5P, MG5PP, Phis, PPHis, and PPPHis) were synthesized (FIGS. 7 to 10). The yield was as follows.
MG5P-PHis: yield 24%
MG5P-PPHis: yield 24%
MG5P-PPPHis: yield 21%
MG5PP-PPPHis: yield 15%

### Test Example 4: Gelation of Polypeptide Solutions

MG5P-PHis, MG5P-PPHis, MG5P-PPPHis, and MG5PP-PPPHis were each dissolved in a 10 w/v % aqueous sucrose solution to a final concentration of 0.5 wt %. The resulting polypeptide solutions were heated to 37° C. and left at that temperature. After 7 days, the polypeptide solutions had turned into clear gels.

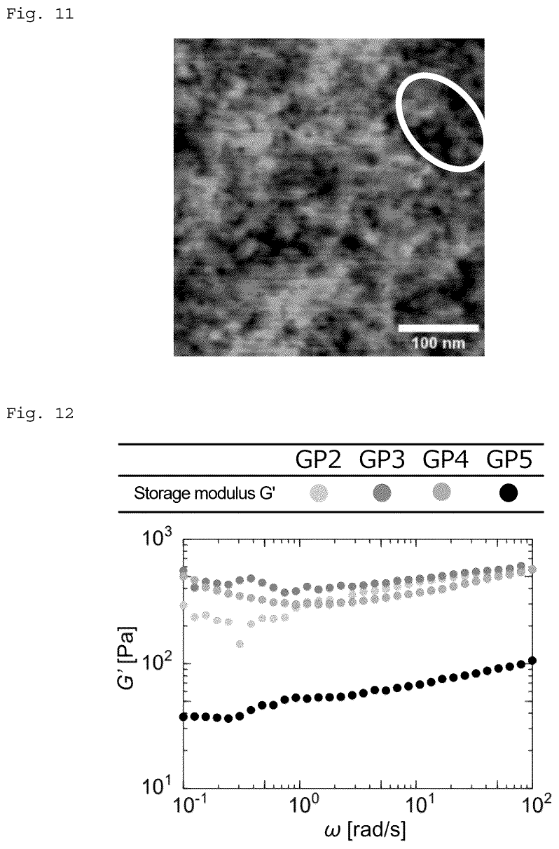

In addition, MG5P-PPHis was dissolved in a 10 w/v % aqueous sucrose solution to a final concentration of 0.1 wt %, and the resulting dispersion was kept at 37° C. for 7 days, then dropped on a mica substrate, and dried at 37° C. When the resulting sample was observed with an atomic force microscope (AFM), an entangled nanofiber structure was observed (FIG. 11).

### Test Example 5: Dynamic Viscoelasticity Measurement

The polypeptide gels obtained in Test Example 4 were each set in an MCR302 rheometer adjusted to 37° C. for dynamic viscoelasticity measurement. The dynamic viscoelasticity was measured in frequency dispersion, from which storage modulus (G′) and loss tangent (tan δ) were determined. The frequency dispersion measurement was performed at a constant strain of 0.1% and in the angular frequency range of 100 to 0.1 rad/s.

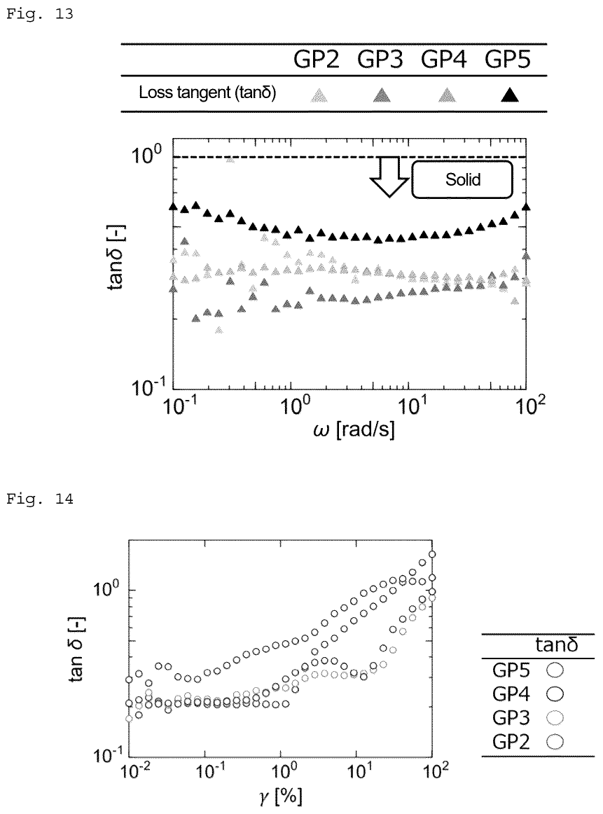

The measurement results of the storage modulus are shown in FIG. 12, and the measurement results of the loss tangent are shown in FIG. 13. FIG. 12 revealed that MG5P-PHis, MG5P-PPHis, and MG5P-PPPHis had higher storage modulus than MG5PP-PPPHis. FIG. 13 revealed that tan δ≤1 held for all samples, regardless of the angular frequency ω. In addition, FIGS. 12 and 13 revealed that MG5P-PPHis had the largest G′ and the smallest tan δ; that is, it formed the hardest and most solid gel.

Furthermore, the strain dispersion measurement of the loss tangent (tan δ) revealed that MG5P-PPHis exhibited solid-like properties even under 100% strain, and that as the strain increased, MG5PP-PPPHis, MG5P-PHis, MG5P-PPPHis, and MG5P-PPHis exhibited liquid-like behavior in this order (FIG. 14).

### Test Example 6: Synthesis 4 of Polypeptide (Solid-Phase Synthesis Method)

Polypeptides with the following amino acid sequences and structures were synthesized by a solid-phase synthesis method in the same manner as in Test Example 1. Only the C-terminal cysteine residues in MPP, MPPP, and MG4PP are represented by a three-letter code, and the other amino acid residues are represented by a one-letter code. In Phis, PPHis, and PPPHis, —NH— on the left side of the N-terminal V (valine residue) is a group derived from the valine residue.

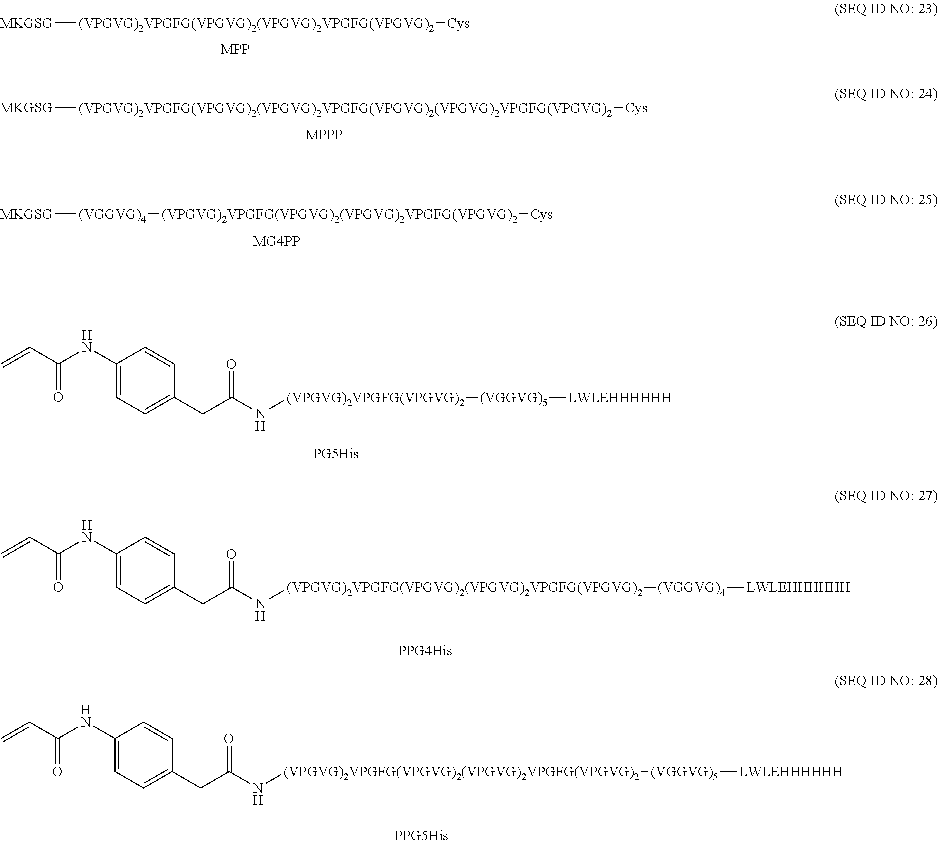

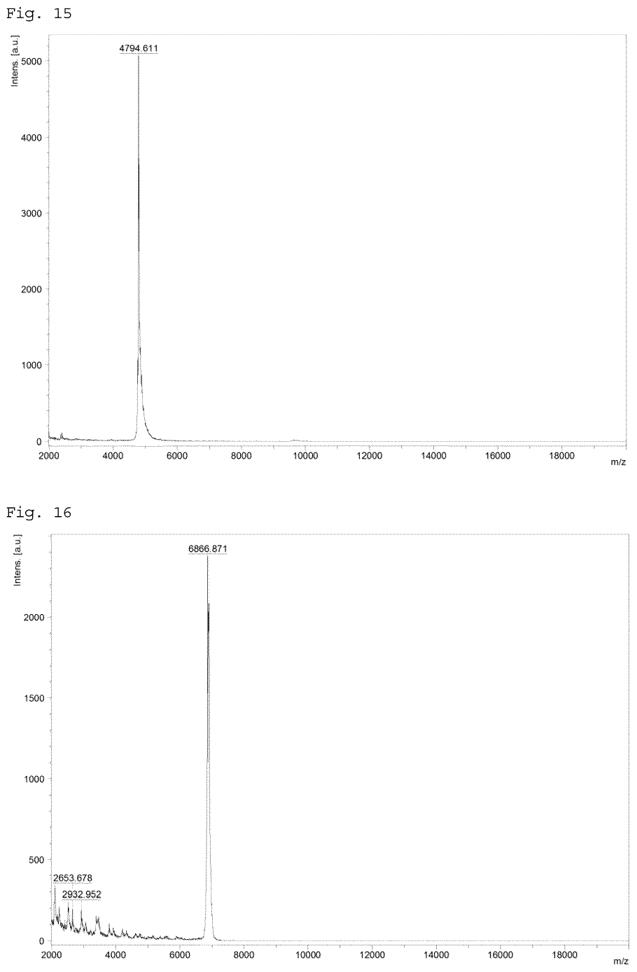

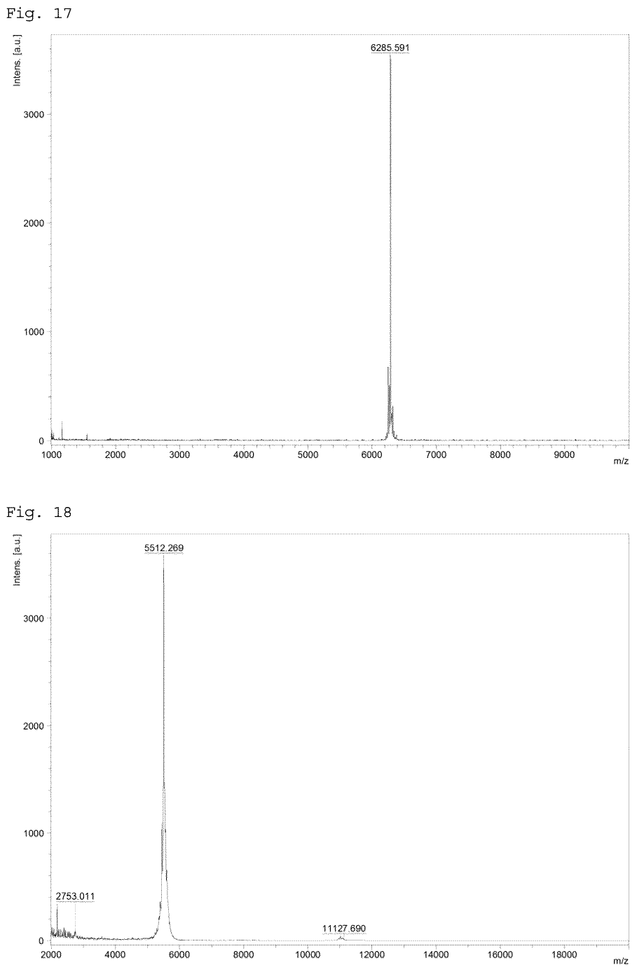

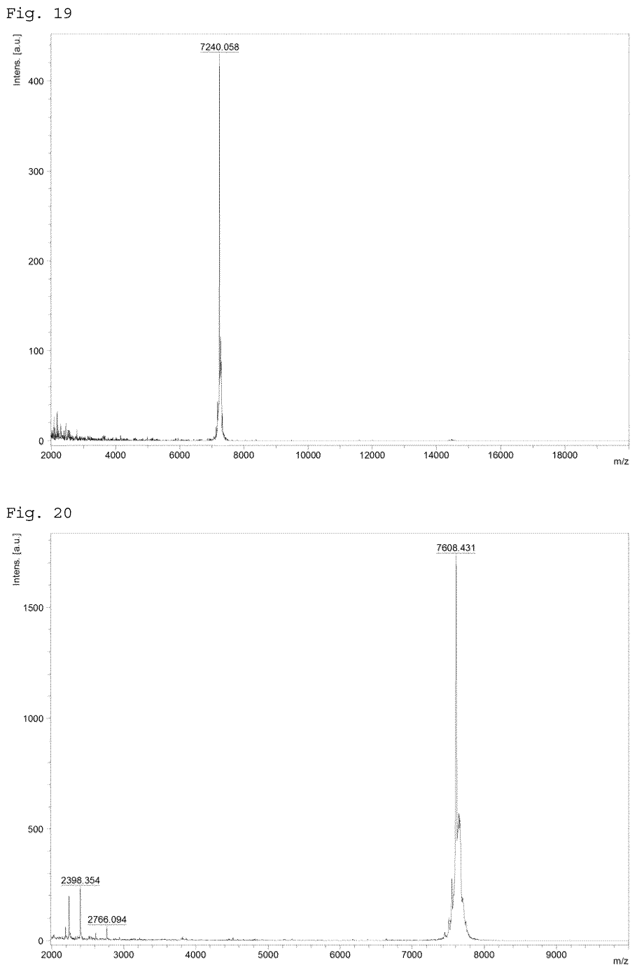

Mass spectrometry confirmed that the target polypeptides (MPP, MPPP, MG4PP, PG5His, PPG4His, and PPG5His) were synthesized (FIGS. 15 to 20). The yield was as follows.
MPP: yield 52%
MPPP: yield 29%
MG4PP: yield 20%
PG5His: yield 17%
PPG4His: yield 10%
PPG5His: yield 6%

### Test Example 7: Synthesis 5 of Polypeptide (Linkage by Chemical Reaction)

Polypeptides with the following amino acid sequences and structures were synthesized.

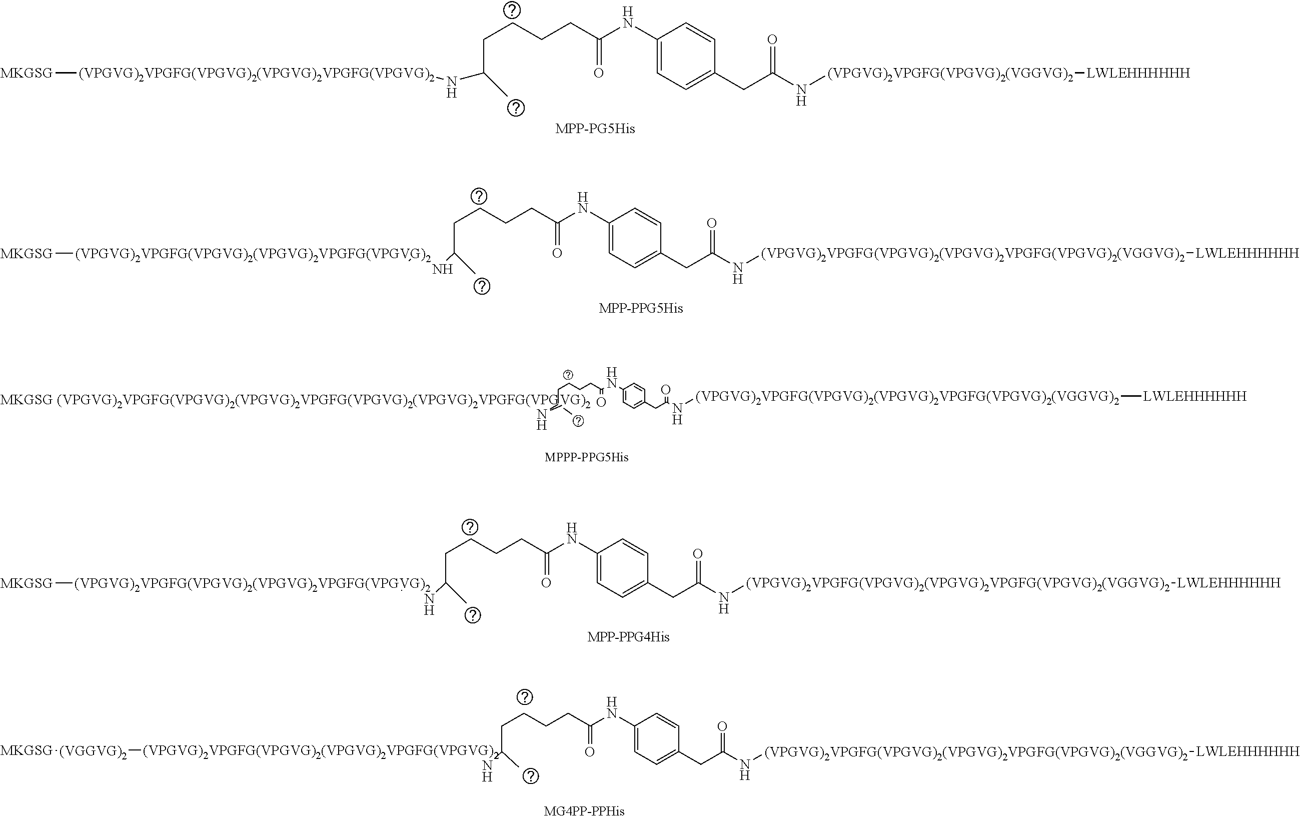

MPP-PG5His was synthesized by linking MPP and PG5His via the thiol-ene reaction. MPP-PPG5His was synthesized by linking MPP and PPG5His via the thiol-ene reaction. MPPP-PPG5His was synthesized by linking MPPP and PPG5His via the thiol-ene reaction. MPP-PPG4His was synthesized by linking MPP and PPG4His via the thiol-ene reaction. MG4PP-PPHis was synthesized by linking MG4PP and PPHis via the thiol-ene reaction.

The thiol-ene reaction was performed as follows. 1.5 molar equivalents of peptide segment 1 (MPP, MPPP, or MG4PP) and 1.0 molar equivalent of peptide segment 2 (PG5His, PPG4His, and PPG5His, or PPHis) were weighed into a vial bottle, and sodium carbonate buffer (pH: 10) was added to adjust the concentration of peptide segment 2 to 10 mM. Dissolved oxygen in the buffer was removed in advance by bubbling nitrogen gas. The reaction solution was heated to 50° C. with stirring and reacted for 18 hours. The reaction was monitored by analytical HPLC. The mixture was then diluted with 50% acetonitrile aqueous solution containing 0.1 v/v % TFA and purified by preparative HPLC.

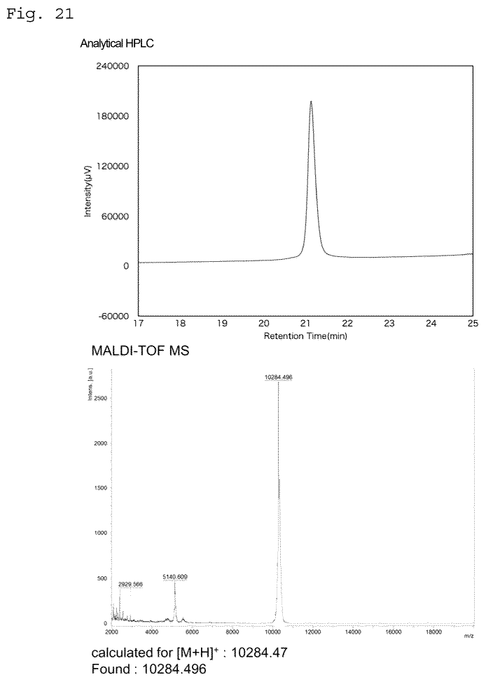

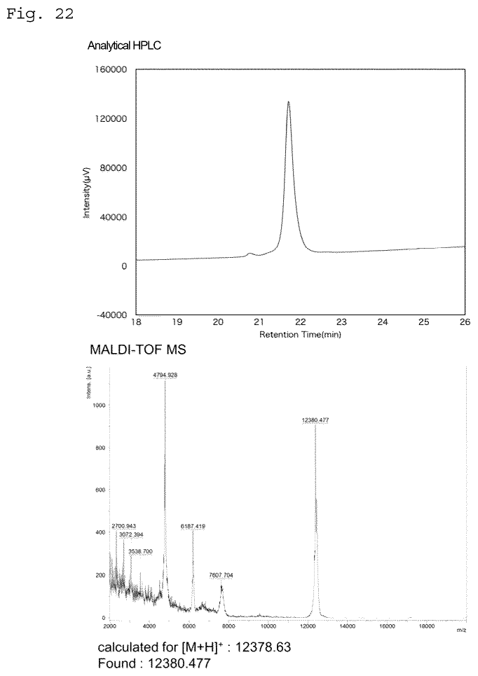

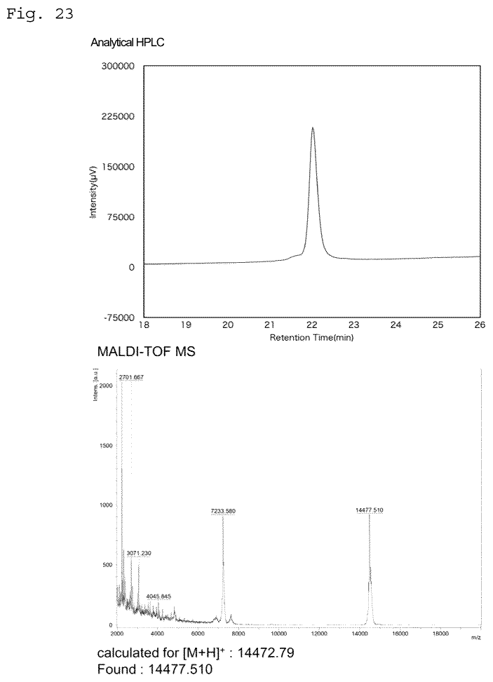

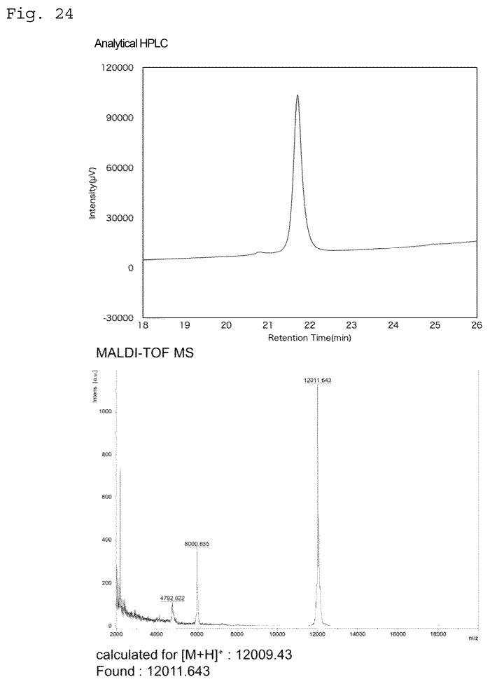

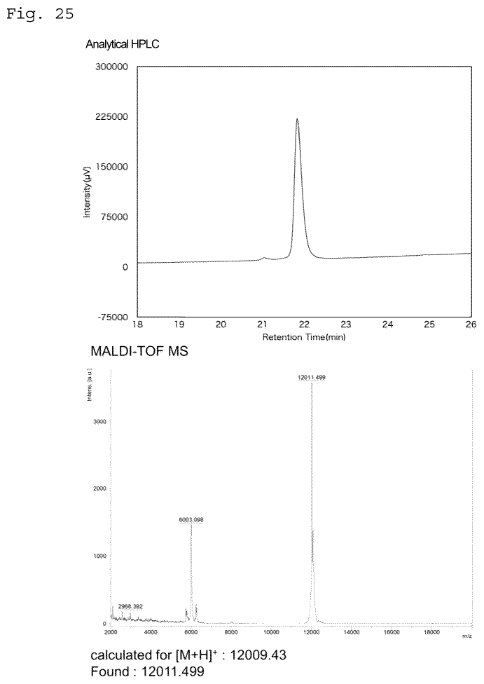

Mass spectrometry confirmed that the target polypeptides (MPP-PG5His, MPP-PPG5His, MPPP-PPG5His, MPP-PPG4His, and MG4PP-PPHis) were synthesized (FIGS. 21 to 25). The yield was as follows.
MPP-PG5His: yield 30%
MPP-PPG5His: yield 23%
MPPP-PPG5His: yield 20%
MPP-PPG4His: yield 20%
MG4PP-PPHis: yield 20%

1. A method for producing a polypeptide comprising an elastin-like block peptide sequence that comprises a G sequence block consisting of (SEQ ID NO: 1: X¹GGX²G)ₙ, wherein each X¹ is the same or different and represents V or L, each X² is the same or different and represents V or L, and n represents an integer of 2 or more, and a P sequence block consisting of (SEQ ID NO: 2: VPGX³G)ₘ, wherein each X³ is the same or different and represents any amino acid, and m represents an integer of 5 or more,
the method comprising:
(a) synthesizing the polypeptide and/or split fragments of the polypeptide by solid-phase synthesis; and/or
(b) linking terminal regions of the multiple split fragments of the polypeptide by a chemical reaction,
wherein when the method comprises step (b), the chemical reaction in step (b) is a reaction between a thiol group of an amino acid residue a in the terminal region of one split fragment a and a vinyl group or a maleimide group added to an amino acid residue b in the terminal region of the other split fragment b.

2. The production method according to claim 1, wherein the sequence structure of the elastin-like block peptide sequence is, from the N-terminal side, G sequence block-P sequence block-G sequence block, G sequence block-P sequence block-P sequence block-G sequence block, P sequence block-G sequence block, or G sequence block-P sequence block.

3. The production method according to claim 1, wherein the method comprises step (a), and the split fragments of the polypeptide each have 30 or more amino acid residues.

4. The production method according to claim 3, wherein the split fragments of the polypeptide each have 50 or more amino acid residues.

5. The production method according to claim 1, wherein the method comprises step (a), and in step (a), the split fragments each contain all or part of the P sequence block.

6. The production method according to claim 1, wherein the method comprises step (a), and the polypeptide and/or each split fragment of the polypeptide contains a hydrophilic amino acid-rich sequence.

7. The production method according to claim 1, wherein the method comprises step (b), and the chemical reaction in step (b) is a reaction between a thiol group of an amino acid residue a in the terminal region of one split fragment a and a vinyl group or a maleimide group added to an amino acid residue b in the terminal region of the other split fragment b.

8. The production method according to claim 7, wherein a group represented by formula (A): 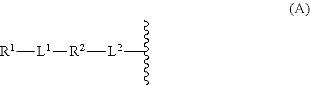
wherein R¹ represents a vinyl group or a maleimide group, L¹ represents a linker containing an amide bond or an ester bond, R² represents an aromatic ring, and L² represents a linker, is added to the terminal region of the split fragment b.

9. The production method according to claim 1, wherein the polypeptide has 80 or more amino acid residues.

10. The production method according to claim 1, wherein the polypeptide comprises an elastin-like block peptide sequence that comprises a G sequence block consisting of (SEQ ID NO: 1: X¹GGX²G)ₙ, wherein each X¹ is the same or different and represents V or L, each X² is the same or different and represents V or L, and n represents an integer of 2 or more, and a P sequence block consisting of (SEQ ID NO: 2: VPGX³G)ₘ, wherein each X³ is the same or different and represents any amino acid, and m represents an integer of 5 or more, and
(c) the sequence structure of the elastin-like block peptide sequence is G sequence block-P sequence block from the N-terminal side, and/or
(d) m is an integer of 5 to 22.

11. A polypeptide comprising an elastin-like block peptide sequence that comprises a G sequence block consisting of (SEQ ID NO: 1: X¹GGX²G)ₙ, wherein each X¹ is the same or different and represents V or L, each X² is the same or different and represents V or L, and n represents an integer of 4 or more, and a P sequence block consisting of (SEQ ID NO: 2: VPGX³G)ₘ, wherein each X³ is the same or different and represents any amino acid, and m represents an integer of 5 or more, and
(c) the sequence structure of the elastin-like block peptide sequence is G sequence block-P sequence block from the N-terminal side, and
(d) m is an integer of 8 to 22.

12. The polypeptide according to claim 11, wherein m is 13 to 22.

13. A gel composition comprising the polypeptide according to claim 11.

14. A gel composition comprising the polypeptide according to claim 12.
